(19) United States
(12) Patent Application Publication
(10) Pub. No.: US 2025/0276005 A1
(43) Pub. Date: Sep. 4, 2025

(54) POLYSACCHARIDES FOR IV ADMINISTRATION THAT TREAT SARS-COV-2 INFECTIONS

(71) Applicant: Pharmalectin, Inc., Needham, MA (US)

(72) Inventor: David PLATT, Newton, MA (US)

(21) Appl. No.: 18/035,582

(22) PCT Filed: Nov. 5, 2021
(86) PCT No.: PCT/US2021/058321

§ 371 Date: May 5, 2023

Related U.S. Application Data
(60) Provisional application No. 63/110,736, filed on Nov. 6, 2020.

(51) Int. Cl. A61K 31/736; A61P 31/14

(52) U.S. Cl. CPC A61K 31/736; A61P 31/14

(57) ABSTRACT

The invention provides a method for treating SARS-COV-2 by administering an effective amount of pectin polysaccharides to a subject in need thereof.

### BACKGROUND OF THE INVENTION

SARS-COV-2 is a single-stranded RNA virus that causes the disease COVID-19. The ongoing SARS-COV-2 pandemic has caused more than 3,000,000 deaths. There are no known treatments that lower the infectivity of SARS-COV-2. Additional treatments for SARS-COV-2 infections that lower the infectivity of SARS-COV-2 are needed.

### SUMMARY OF THE INVENTION

The present invention provides a method of treating a SARS-COV-2 infection in a subject in need thereof, by administering to the subject an effective amount of pectin polysaccharides, or a pharmaceutical composition thereof.

In some embodiments of the method of the invention, administration of the effective amount of pectin polysaccharides, or a pharmaceutical composition thereof, reduces the SARS-COV-2 infectivity of a subject relative to the SARS-COV-2 infectivity of the subject in the absence of pectin polysaccharide administration.

In some embodiments of the method of the invention, administration of the effective amount of pectin polysaccharides, or a pharmaceutical composition thereof, decreases the copy number of SARS-COV-2 RNA polynucleotides present in a biological sample from the subject, e.g., the nasal secretions, blood, saliva, sputum, serum, and/or stool of the subject, relative to the copy number of SARS-COV-2 RNA polynucleotides present in a biological sample of the subject in the absence of pectin polysaccharide administration, e.g., prior to administration.

In some embodiments of the method of the invention, administration of the effective amount of pectin polysaccharides, or a pharmaceutical composition thereof, increases the cycle threshold (Ct) number of a SARS-COV-2 gene, e.g., the envelope protein (E) gene, nucleocapsid (N) gene, and/or the Ct number of the SARS-COV-2 RNA-dependent RNA polymerase (Rd/Rp) gene, e.g., measured in a real time polymerase chain reaction (RT-PCR) experiment, e.g., using a biological sample obtained from the subject, e.g., nasal secretions, blood, saliva, sputum, serum, and/or stool, relative to the Ct number of the gene in the absence of pectin polysaccharide administration, e.g., prior to administration.

In some embodiments, the pectin polysaccharides are administered to the subject at least one time per day, e.g., at least two times per day, at least three times per day, at least five times per day, at least six times per day, at least seven times per day, at least eight times per day, at least nine times per day, at least ten times per day, e.g., up to 10 times a day. In some embodiments, the pectin polysaccharides, or pharmaceutical composition thereof, is administered to the subject one time per hour during each hour that the subject is awake, e.g., up to 10 times a day.

In some embodiments, the infection is a symptomatic invention. In some embodiments, the infection is an asymptomatic infection. In some embodiments, the infection is an rRT-PCR positive infection. In some embodiments, the infection is a mild infection. In some embodiments, the infection is a moderate infection. In some embodiments, the infection is a severe infection.

In some embodiments of the method of the invention, the pectin polysaccharides are administered intravenously.

In some embodiments, the method of the invention results in a higher Immunoglobulin G (IgG) antibody titer in the subject relative to the IgG antibody titer observed in the absence of pectin polysaccharide administration, e.g., prior to administration.

In a second aspect, the present invention provides a method for reducing the number of SARS-COV-2 virons in a sample of cells, by contacting the sample with an effective amount of pectin polysaccharides.

In some embodiments, the number of virons in the sample of cells is reduced by at least 50%, e.g., at least 60%, at least 70%, at least 80%, at least 90%, at least 95%, at least 99%. In some embodiments, the sample of cells is substantially free of virons following being contacted with the effective amount of pectin polysaccharides.

In some embodiments, the sample of cells is exposed to SARS-COV-2 virons prior to being contacted with the effective amount of pectin polysaccharides. In some embodiments, the cells are contacted with the effective amount of pectin polysaccharides prior to being exposed to SARS-COV-2 virons.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the term “approximately” may be understood to permit standard variation as would be understood by those of ordinary skill in the art; and (v) where ranges are provided, endpoints are included.

As used herein, the term “about” represents a value that is in the range of +10% of the value that follows the term “about.” Reference to “about” a value or parameter herein includes (and describes) variations that are directed to that value or parameter per se. For example, description referring to “about X” includes description of “X”.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, and vitreal.

As used herein, the term “biological sample” is a sample obtained from a subject including but not limited to blood (e.g., whole blood, processed whole blood (e.g., a crude whole blood lysate), serum, plasma, and other blood derivatives), bloody fluids (e.g., wound exudate, phlegm, bile, and the like), cerebrospinal fluid (CSF), urine, synovial fluid, breast milk, sweat, tears, saliva, semen, feces, vaginal fluid or tissue, sputum (e.g., purulent sputum and bloody sputum), nasopharyngeal aspirate or swab, lacrimal fluid, mucous, or epithelial swab (buccal swab), tissues (e.g., tissue biopsies (e.g., skin biopsies (e.g., from wounds, burns, or tick bites), muscle biopsies, or lymph node biopsies)), including tissue homogenates), organs, bones, teeth, among others. In some embodiments, the biological sample contains cells and/or cell debris derived from the subject from which the sample was obtained. In some embodiments, the subject is a host of a pathogen, e.g., SARS-COV-2, and the biological sample obtained from the subject includes subject (host)-derived cells and/or cell debris, as well as one or more pathogen cells or viral particles, e.g., SARS-COV-2 viral particles. In some embodiments, the biological sample contains nucleic acids, e.g., DNA and/or RNA, derived from the subject from which the sample was obtained, as well as nucleic acids derived from the pathogen cells or viral particles, e.g., nucleic acids derived from SARS-COV-2.

A “symptomatic infection” indicates the subject infected with SARS-COV-2 has one or more symptoms of SARS-COV-2 infection including, but not limited to: fever, chills, cough, shortness of breath or difficulty breathing, fatigue, muscle or body aches, confusion, headache, inability to wake up or stay awake, pale, gray, or blue-colored skin, lips, or nail beds, loss of taste or smell, sore throat, congestion or runny nose, nausea, vomiting, or diarrhea.

An “asymptomatic infection” indicates the subject infected with SARS-COV-2 has not developed any symptoms of SARS-COV-2 infection. An asymptomatic infection includes both subjects who later go on to develop one or more symptoms, and subjects who never develop one or more symptoms.

A “rRT-PCR positive infection” indicates that the subject infected with SARS-COV-2 has developed a sufficient viral load for viral RNA polynucleotides to be detected by a real-time reverse transcription polymerase chain reaction test.

A “mild infection” indicates that the subject infected with SARS-COV-2 experiences symptoms including a fever equal to or below about 100 degrees Fahrenheit, a cough, chills, fatigue, muscle or body aches, confusion, headache, loss of taste or smell, sore throat, congestion, runny nose, nausea, vomiting, or diarrhea.

A “moderate infection” indicates that the subject infected with SARS-COV-2 experiences symptoms including a fever above about 100 degrees Fahrenheit, some shortness of breath, or chills with repeated shaking, in addition to any of the symptoms experienced by a subject experiencing a mild SARS-COV-2 infection.

A “severe infection” indicates that the subject infected with SARS-COV-2 experiences symptoms including shortness of breath or difficulty breathing; inability to wake up or stay awake; pale, gray, or blue-colored skin, lips or nail beds; or persistent pain and pressure in chest; in addition to any of the symptoms experienced by a subject experiencing a mild or moderate SARS-COV-2 infection.

As used herein, the Cycle threshold (Ct number) is the number of PCR cycles required for the fluorescent signal caused by the production of a particular rRT-PCR product to exceed a predetermined threshold.

An “effective amount” of a compound may vary according to factors such as the disease state, age, sex, and weight of the individual, and the ability of the compound to elicit the desired response. A therapeutically effective amount encompasses an amount in which any toxic or detrimental effects of the compound are outweighed by the therapeutically beneficial effects. An effective amount also encompasses an amount sufficient to confer benefit, e.g., clinical benefit.

As used herein, “infectivity” refers to the ability of an individual infected with SARS-COV-2 to transmit the infection to another individual. The infectivity of an individual is determined by the level of SARS-COV-2 virons in the individual, as measured by the Ct values expressed for the SARS-COV-2 RNA polymerase (Rd/RP) gene, nucleocapsid (N) gene, and/or envelope (E) gene in a sample (e.g., a nasopharyngeal swab, a saliva sample, a stool sample, a serum sample, a blood sample) obtained from the patient.

The term “pharmaceutical composition,” as used herein, represents a composition containing a compound described herein formulated with a pharmaceutically acceptable excipient. Pharmaceutical compositions can be formulated, for example, for oral administration in unit dosage form (e.g., a tablet, capsule, caplet, gel cap, suspension, solution, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

As used herein, the term “subject” or “participant” or “patient” refers to any organism to which a compound or composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventive measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the subject; or enhancement or improvement of condition, disorder, or disease. Treatment includes eliciting a clinically significant response without excessive levels of side effects. Treatment also includes prolonging survival as compared to expected survival if not receiving treatment.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIG. 1 shows the structure of pectin polysaccharides employed in the present invention.

FIG. 2 shows the structure of pectin polysaccharides employed in the present invention.

FIG. 3 is a flow chart showing an experimental protocol used to measure the effect of pectin polysaccharides on the viral load of SARS-COV-2 in Vero cells (ATCC® CCL-81™).

FIG. 4 is a chart showing reduction in viral particles present in a sample of Vero cells (ATCC® CCL-81™) following administration of pectin polysaccharides.

FIG. 5 is a chart showing reduction in viral particles present in a sample of Vero cells (ATCCR CCL-81™) following administration of pectin polysaccharides.

FIG. 6 is a total ion chromatograph showing the results of gas chromatography/mass spectrometry analysis of partially methylated alditol acetate derivatives made from a sample of pectin polysaccharides.

FIG. 7 is a full range ¹H NMR spectrum of pectin polysaccharides.

FIG. 8 is a ¹H NMR spectrum of the carbohydrate region of pectin polysaccharides.

FIG. 9 is a HSQC spectrum of the anomeric region of pectin polysaccharides.

FIG. 10 is a HSQC spectrum of the glycosyl ring-H/C region of pectin polysaccharides.

FIG. 11 is a TOCSY spectrum of pectin polysaccharides.

### DETAILED DESCRIPTION

Treatment options for SARS-COV-2 infections are limited. In particular, there are no treatments available that limit or decrease the infectivity of SARS-COV-2. The present inventors have discovered that pectin polysaccharides are an effective treatment for SARS-COV-2. The present inventors have further discovered that pectin polysaccharides are effective at decreasing the infectivity of SARS-COV-2. Therefore, one object of this invention is to provide a method for treating SARS-COV-2 by administering an effective amount of pectin polysaccharides to a subjected in need thereof. A second of object of this invention is to provide a method for treating SARS-COV-2 that limits the infectivity of SARS-COV-2, by administering an effective amount of pectin polysaccharides to a subject in need thereof. A third object of this invention is to provide a method for decreasing the number of SARS-COV-2 virons in a sample of cells, by contacting the sample with an effective amount of pectin polysaccharides.

### SARS-COV-2

SARS-COV-2 is a single-stranded RNA virus that causes the disease COVID-19. Nonlimiting examples of symptoms of COVID-19 infections include fever, cough, headache, fatigue, breathing difficulties, nasal congestion and runny nose, sore throat, diarrhea, and loss of smell and taste. The majority of individuals who suffer from COVID infections experience mild or moderate symptoms. However, approximately 15% of individuals who become infected with SARS-COV-2 experience severe symptoms. Nonlimiting examples of severe symptoms include dyspnea, hypoxia, respiratory failure, shock, multiorgan dysfunction, or death. A subset of patients who become infected with SARS-COV-2 experience “long-haul infections” in which COVID-19 symptoms including but not limited to fatigue, headaches, shortness of breath, loss of smell, muscle weakness, low fever, and cognitive dysfunction continue for a period of time (e.g., days, weeks, months) following their diagnosis.

COVID-19 transmission is thought to occur mainly through respiratory route via SARS-COV-2 virions that are contained in the respiratory droplets and/or aerosols of individuals infected with COVID-19. Transmission occurs when the respiratory droplets or aerosols enter the mouth, nose, or eyes of a second individual. Approximately 1,000 COVID virons are believed to be sufficient to initiate a new infection.

### Detection of SARS-COV-2 in Biological Samples

SARS-COV-2 infections are commonly diagnosed by detection of the viral nucleic acids via real-time reverse transcription polymerase chain reaction (rRT-PCR). Samples for the diagnosis of a COVID-19 infection may be nasal secretions, (e.g., material obtained from the nasal passages and/or sinuses, optionally via nasopharyngeal swab), or blood, saliva, sputum, stool, or serum of a subject. rRT-PCR experiments involve monitoring the amplification of a nucleic acid, e.g., a nucleic acid produced by SARS-CoV-2, during a PCR experiment.

PCR experiments involve repeated cycles of heating and cooling a buffered mixture, e.g., of nucleotide triphosphates, a polynucleotide from a sample of interest, a probe polynucleotide with a sequence complementary to a portion of the nucleotide sequence in the sample that is intended to be amplified (the target sequence), and enzymes capable of catalyzing the chemical reactions that lead to the amplification of the sequence. In rRT-PCR experiments, the progress of polynucleotide amplification reactions is indirectly measured by observing changes in fluorescence that occur upon binding of fluorophore-containing probe nucleotides to nucleotides containing the target sequence. Target sequences present in high concentrations in a sample analyzed by rRT-PCR will have lower Ct values than target sequences present in low concentrations in rRT-PCR samples.

Multiple nucleotide molecules produced by SARS-COV-2 may be detected using rRT-PCR. Nonlimiting examples of nucleotide molecules produced by SARS-COV-2 that may be detected via rRT-PCR include the portion of the SARS-COV-2 RNA genome encoding the envelope protein (the E gene) the portion of the SARS-COV-2 RNA genome encoding the nucleocapsid protein (the N gene), and the RdRP gene, the portion of the SARS-COV-2 RNA genome encoding the RNA dependent RNA polymerase protein (the Rd/RP gene), may be detected using rRT-PCR. In some embodiments, the E gene, N gene, and Rd/Rp gene are detected individually. In some embodiments, multiple genes are detected in a single rRT-PCR experiment (e.g., the Rd/Rp gene+the N gene).

The Ct value measured for a given gene in a biological sample is related to the copy number of the gene (i.e., the number of copies of the gene present in the sample). The copy number of the SARS-COV-2 E gene, N gene, and/or Rd/RP gene in a specimen (e.g., a sample of nasal secretions, a blood sample, a stool sample, a saliva sample, a sputum sample, a serum sample, the lysate of a sample of cells, a sample of cell supernatant) may be calculated from the Ct value of the gene in the sample.

The infectivity of a subject infected with SARS-COV-2 is related to the quantity of SARS-COV-2 virons present in the subject (e.g., the viral load of the subject). The copy number of SARS-COV-2 E gene, N gene, and/or Rd/RP gene in a sample (e.g., a sample of nasal secretions, a blood sample, a stool sample, a saliva sample, a sputum sample, a serum sample) obtained from a subject infected with SARS-COV-2 is related to the number of SARS-COV-2 virons present in the subject. A subject with a higher number of SARS-COV-2 virons in their body (e.g., in their nasal secretions, blood, stool, saliva, sputum, serum) is likelier to transmit a SARS-COV-2 infection than a subject with a lower number of SARS-COV-2 virons in their body. Thus, the infectivity of a subject infected with SARS-COV-2 can be measured via the Ct value of the SARS-COV-2 E gene, N gene, and/or RdRp gene measured in a sample (e.g., a sample of nasal secretions, a blood sample, a stool sample, a saliva sample, a sputum sample, a serum sample) obtained from the subject.

### Pectin Polysaccharides

Pectin polysaccharides are complex, heterogeneous, glycans that can be derived from derived from crude biomass and that include terminal arabinofuranosyl residues, terminal arabinopyranosyl residues, 2-linked rhamnopyranosyl residues, terminal galactopyranosyl residues, terminal galactopyranosyl uronic acid residues, 2-linked xylopyranosyl residues, 4-linked xylopyranosyl residues, 2,4-linked rhamnopyranosyl residues, 2,4-linked rhamnopyranosyl residues, 3-linked galactopyranosyl residues, 4-linked galactopyranosyl residues, 4-linked galactopyranosyl uronic acid residues, 4-linked glucopyranosyl residues, 3,4-linked galactopyranosyl uronic acid residues, and/or 3,5-linked galactopyranosyl residues.

Pectin polysaccharides may be obtained via the processing of crude fruit pectins, e.g., apple pectins, e.g., pectins derived from apple pomace, or citrus pectins, e.g., pectins derived from citrus peels, e.g., the peels of oranges, lemons, or limes, or from the processing of soybean pectins, e.g., pectins derived from soybean hulls, or sugar beet pectins, e.g., pectins derived from sugar beets. In some embodiments, Pectin polysaccharides are derived from apple pomace. In some embodiments, the pectin polysaccharides is obtained through chemical, enzymatic, physical treatment, and purification from pectic substance of citrus peels and apple pomace or soybean hull or alternatively processed from sugar beet pectin, e.g., as described in U.S. Pat. No. 10,744,154, which is hereby incorporated by reference. An exemplary pectin polysaccharide is Prolectin-I, as described herein.

Although the composition of pectin may vary among plants, pectin typically has a composition in which D-galacturonic acid is the main monomeric constituent. The D-galacturonic residues of pectin optionally may be substituted with D-xylose or D-apiose to form xylogalacturonan and apiogalacturonan, respectively, branching from a D-galacturonic acid residue. So-called “rhamnogalcturonan pectins” contain a backbone of repeating disaccharides of D-galacturonic acid and L-rhamnose.

In one some embodiments, pectin polysaccharides are prepared by modifying naturally occurring polymers to reduce the molecular weight for the desired range, reducing the alkylated group (de-methoxylation or de acetylation). Prior to chemical modification, the natural polysaccharides may have a molecular weight range of between about 40,000-1,000,000 with multiple branches of saccharides, for example, branches comprised of 1 to 20 monosaccharides of glucose, arabinose, galactose etc, and these branches may be connected to the backbone via neutral monosaccharides such as rhamnose. These molecules may further include a single or chain of uronic acid saccharide backbone that may be esterified from as little as about 2% to as much as about 30%. The multiple branches themselves may have multiple branches of saccharides, the multiple branches optionally including neutral saccharides and neutral saccharide derivatives creating mainly hydrophobic entities.

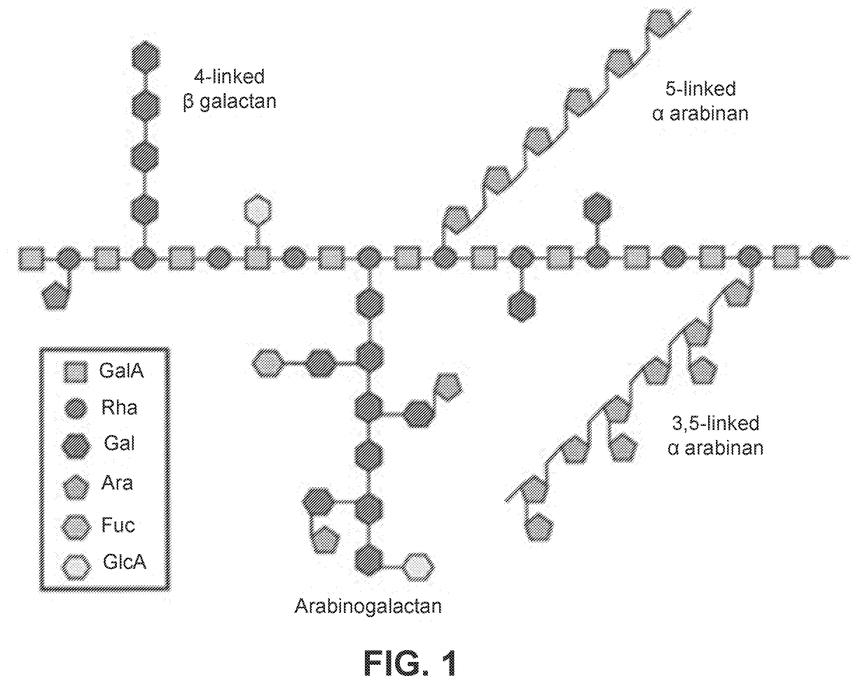

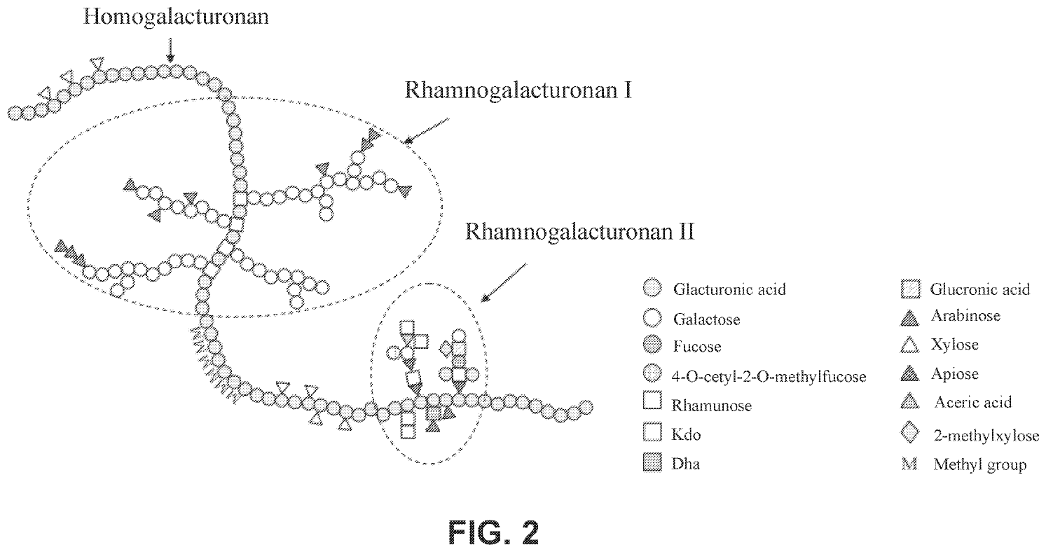

In some embodiments, the pectin polysaccharides have the structures shown in FIGS. 1-2.

In some embodiments, pectin polysaccharides have a weight-average molecular weight of about 40 kDa to about 1 MDa, e.g., 50 kDa to about 500 kDa, about 60 kDa to about 400 kDa, about 70 kDa to about 300 kDa, about 80 kDa to about 200 kDa, about 90 kDa to about 150 kDa, about 100 kDa to about 140 kDa, about 110 kDa to about 130 kDa, or about 120 kDa. In some embodiments, pectin polysaccharides have a weight average molecular weight of about 120 kDa.

In some embodiments, pectin polysaccharides have a heterogeneous structure with five principal components: rhamnose, fucose, arabinose, galactose, and uronate. In some embodiments, pectin polysaccharides are about 1% to about 10% rhamnose by weight, e.g., about 2% to about 8%, about 3% to about 7%, about 4% to about 6%, about 4.3% rhamnose by weight; about 1% to about 10% fucose by weight, e.g., about 2% to about 6%, about 3% to about 5%, about 3.7% fucose by weight; about 10% to about 30% arabinose by weight, e.g., about 12% to about 28%, about 14% to about 26%, about 16% to about 24%, about 18% to about 22%, about 19% arabinose by weight; about 30% to about 50% galactose by weight, e.g., about 32% to about 46%, about 34% to about 42%, about 36% to about 48%, about 37% galactose by weight; and about 25% to about 45% uronate by weight, e.g., about 27% to about 43%, about 29% to about 41%, about 31% to about 39%, about 33% to about 37%, about 36% uronate by weight. In some embodiments, the backbone of pectin polysaccharides is mainly composed of α-(1,2)-L-rhamnosyl-α-(1,4)-D-galacturonosyl sections.

In some embodiments, pectin polysaccharides are a branched heteropolymer of alternating a-1,2-linked rhamnose and alpha-1,4-linked Gala residues that carries neutral side-chains of predominantly 1,4-beta-D-galactose and/or 1,5-alpha-L-arabinose residues attached to the rhamnose residues of the RGI backbone. GR side-chains may be decorated with arabinosyl residues (arabinogalactan I) or other sugars, including fucose, xylose, and mannose.

In some embodiments, the pectin polysaccharides of the present invention bind to SARS-COV-2 virons by a specified amount. Contacting a sample of SARS-COV-2 virons, e.g., a sample of SARS-COV-2 virons in a sample of cells (e.g., Vero (African green monkey kidney cells, Vero (ATTC® CCL-81™))) or a sample of SARS-COV-2 virons in a biological sample, e.g., a biological sample obtained from a subject infected with SARS-COV-2, with the pectin polysaccharides of the invention results in a decrease in the quantity of SARS-COV-2 virons in the sample, e.g., as determined by a rRT-PCR experiment. Contacting the sample of SARS-COV-2 virons with the pectin polysaccharides of the invention results in at least an about 80% reduction in the quantity of SARS-COV-2 virons, e.g., an about 80% reduction an about 81% reduction, an about 82% reduction, an about 83% reduction, an about 84% reduction, an about 85% reduction, an about 86% reduction, an about 87% reduction, an about 88% reduction, an about 89% reduction, an about 90% reduction, an about 91% reduction, an about 92% reduction, an about 93% reduction, an about 94% reduction, an about 95% reduction, an about 96% reduction, an about 97% reduction, an about 98% reduction, an about 99% reduction, an about 100% reduction of the quantity of SARS-COV-2 virons in the sample of SARS-COV-2 virons, e.g., as determined by a rRT-PCT experiment. The following description of pectin polysaccharide activity is provided without wishing to be bound by theory.

The N-terminal domain (NTD) of the SARS-COV-2 spike protein is essential for vial entry into human cells. Portions of the SARS-COV-2 spike protein resemble human galectins, which contain a highly conserved carbohydrate-binding domain that binds a variety of pectin polysaccharides. In some embodiments, the pectin polysaccharides bind to the NTD of the SARS-COV-2 spike protein. In some embodiments, binding of pectin polysaccharides to the NTD of the SARS-COV-2 spike protein is deleterious to the ability of SARS-COV-2 to enter a human cell.

In some embodiments, pectin polysaccharides bind to galectins on the surface of the SARS-COV-2 viral particle that are not the NTD of the SARS-COV-2 spike protein. In some embodiments, pectin polysaccharides prevent entry of SARS-COV-2 virons into human cells by allosterically inhibiting galectins on the surface of the SARS-COV-2 envelope. In some embodiments, pectin polysaccharides bind to galectins on the surface of human cells. In some embodiments, the binding of pectin polysaccharides to galectins on the surface of human cells is deleterious to the ability of SARS-COV-2 to enter human cells. In some embodiments, pectin polysaccharides bind to carbohydrates that are displayed on the surface of the SARS-CoV-2 viral particle. In some embodiments, the binding of pectin polysaccharides to carbohydrates displayed on the surface of the SARS-COV-2 viral particle is deleterious to the ability of SARS-COV-2 to enter human cells. In some embodiments, pectin polysaccharides recruit elements of the immune system, (e.g., leukocytes) to SARS-COV-2 particles. In some embodiments, pectin polysaccharides deactivate SARS-COV-2 particles, which are then eliminated by the liver.

In some embodiments, pectin polysaccharides bind to galectins on viruses other than SARS-COV-2. In some embodiments, pectin polysaccharide binding prevents the entry of viruses other than SARS-COV-2, e.g., adenoviruses or retroviruses, into human cells by allosterically inhibiting galectins on the surface of the viruses.

In some embodiments, the binding of pectin polysaccharides to galectins on the surface of human cells is deleterious for the ability of viruses other than SARS-COV-2 to enter human cells.

In some embodiments, pectin polysaccharides bind to carbohydrates that are displayed on the surface of viruses other than SARS-COV-2 (e.g., adenoviruses or retroviruses). In some embodiments, the binding of the carbohydrates is deleterious to the ability of the viruses to enter human cells.

In some embodiments, pectin polysaccharides recruit elements of the immune system, (e.g., leukocytes) to viral particles other than SARS-COV-2 particles. In some embodiments, pectin polysaccharides deactivate the viral particles, which are then eliminated by the liver.

In some embodiments, pectin polysaccharides stimulate the immune response against SARS-COV-2 in a subject. One element of the immune response of production of immunoglobulin G (IgG). In some embodiments, administration of pectin polysaccharides results in a higher IgG antibody titer in the subject relative to the IgG antibody titer observed in the absence of pectin polysaccharide administration.

### Dosages

The dosage of the composition used in the methods described herein can vary depending on many factors, e.g., the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The composition used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

While the attending physician ultimately will decide the appropriate amount and dosage regiment, in some embodiments an effective amount may about 10 mg/m², about 20 mg/m², about 40 mg/m², about 80 mg/m², or about 160 mg/m². In some embodiments, an effective amount may be between about 10 mg/m² and about 160 mg/m², between about 20 mg/m² and about 100 mg/m², between about 30 mg/m² and about 50 mg/m², or between about 35 mg/m² and about 45 mg/m². In some embodiments, an effective amount may be 10 mg/m² to 160 mg/m², 20 mg/m² to 100 mg/m², 30 mg/m² to 50 mg/m², or 35 mg/m² to 45 mg/m².

### Pharmaceutical Compositions

The administration of pectin polysaccharides may be by any suitable means that results in treatment of a SARS-COV-2 infection. Pectin polysaccharides may be contained in any appropriate amount in any suitable carrier substance and is generally present in an amount of 1-95% by weight of the total weight of the composition. The composition may be provided in a dosage form that is suitable for the sublingual, buccal, oral, parenteral (e.g., intravenously, intramuscularly), pulmonary, intranasal, transdermal, vaginal, or rectal administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, sprays, vapors, or aerosols. The pharmaceutical compositions may be formulated according to conventional pharmaceutical practice (see, e.g., Remington: The Science and Practice of Pharmacy, (23rd ed.) ed. A. Adejare., 2020, Academic Press, Philadelphia, PA).

In some embodiments, pectin polysaccharides are formulated into a solution for IV administration. In some embodiments, pectin polysaccharides are administered intravenously as a continuous infusion. In some embodiments, pectin polysaccharides are administered intravenously as a bolus. In some embodiments, pectin polysaccharides are formulated in a solution for intravenous administration at a concentration of about 0.1 mg/mL, about 0.5 mg/mL, about 1 mg/mL, about 2 mg/mL, about 4 mg/mL, or about 8 mg/mL. In some embodiments, pectin polysaccharides are formulated in a solution for intravenous administration at a concentration of about 0.1 mg/mL-about 8 mg/mL, e.g., about 0.5 mg/ml-about 4 mg/mL, or about 1 mg/mL-about 2 mg/mL.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the active compound within the body over an extended period of time; (ii) formulations that after a predetermined lag time create a substantially constant concentration of the active compound within the body over an extended period of time; and (iii) formulations that sustain active compound action during a predetermined time period by maintaining a relatively, constant, effective active compound level in the body with concomitant minimization of undesirable side effects associated with fluctuations in the plasma level of the active compound (sawtooth kinetic pattern).

Any one of a number of strategies can be pursued in order to obtain controlled release of the active compound in question. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the active compound in a controlled manner. Examples include single or multiple unit tablet or capsule compositions, oil solutions, suspensions, emulsions, microcapsules, microspheres, nanoparticles, patches, and liposomes.

### Methods of Treatment

Pectin polysaccharides, or pharmaceutical compositions thereof, may serve as a useful therapeutic for SARS-COV-2 infections. In particular, pectin polysaccharides, may be useful in treating the symptoms of SARS-COV-2 infection in a subject. In some embodiments, the subject is an adult (e.g., the subject is greater than 18 years old). In some embodiments, the subject is a child (e.g. the subject is less than 18 years old, less than 17 years old, less than 16 years old, less than 15 years old, less than 14 years old, less than 13 years old, less than 12 years old, less than 11 years old, less than 10 years old, less than 9 years old, less than 8 years old, less than 7 years old, less than 6 years old, less than 5 years old, less than 4 years old, less than 3 years old, less than 2 years old, less than 1 year old).

In some embodiments, the pectin polysaccharides are administered fewer than 48 hours following the diagnosis of a SARS-COV-2 infection in the subject (e.g., fewer than 24 hours following the diagnosis of a SARS-COV-2 infection in the subject, fewer than 12 hours following the diagnosis of a SARS-COV-2 infection in the subject, less than 6 hours following the diagnosis of a SARS-COV-2 infection in the subject, less than 3 hours following the diagnosis of a SARS-COV-2 infection in the subject, at substantially the same time as a SARS-COV-2 infection is diagnosed in the subject).

In some embodiments, including any of the foregoing embodiments, the pectin polysaccharides are administered more than 30 minutes after the subject consumes food, e.g., more than 60 minutes, more than 90 minutes, or more than 120 minutes after the subject consumes food.

In some embodiments, pectin polysaccharides are administered to the subject at least once per day. In some embodiments, including any of the foregoing embodiments, pectin polysaccharides are administered to the subject at least twice per day. In some embodiments, including any of the foregoing embodiments, pectin polysaccharides is administered to the subject at least three times per day. In some embodiments, including any of the foregoing embodiments, pectin polysaccharides is administered to the subject at least four times per day. In some embodiments, including any of the foregoing embodiments, pectin polysaccharides are administered to the subject at least five times per day. In some embodiments, including any of the foregoing embodiments, pectin polysaccharides are administered to the subject at least six times per day. In some embodiments, including any of the foregoing embodiments, pectin polysaccharides are administered to the subject at least seven times per day. In some embodiments, including any of the foregoing embodiments, pectin polysaccharides are administered to the subject at least eight times per day. In some embodiments, including any of the foregoing embodiments, pectin polysaccharides are administered to the subject at least nine times per day. In some embodiments, including any of the foregoing embodiments, pectin polysaccharides are administered to the subject at least ten times per day. In some embodiments, including any of the foregoing embodiments, pectin polysaccharides are administered to the subject at least eleven times per day. In some embodiments, including any of the foregoing embodiments, pectin polysaccharides are administered to the subject at least twelve times per day. In some embodiments, including any of the forgoing embodiments, the method comprises administering to the subject pectin polysaccharides hourly, e.g., during waking hours.

### EXAMPLES

### Example 1: Antiviral Testing-SARS-COV-2

### Test(s) Performed

In-vitro assay to demonstrate inhibition of a patient sample derived strain of SARS-COV-2 virus infection of Vero (African green monkey kidney cells, Vero (ATTC® CCL-81™)

### Objective(s) of the Test(s)

To test the viral inhibition effect of the given compounds (blinded) ProLectin-I. 72 hours after infection. The end point was % viral reduction. Viral reduction was defined as:

$$\% Viral Reduction = \frac{\begin{matrix}number of viral particles in infection control - \\ number of viral particles exposed\end{matrix}}{number of viral particles in infection control}$$

### A Brief Description of the Test Methods, Including Sample Size, Device(s) Tested, and Any Consensus Standard(s) Utilized

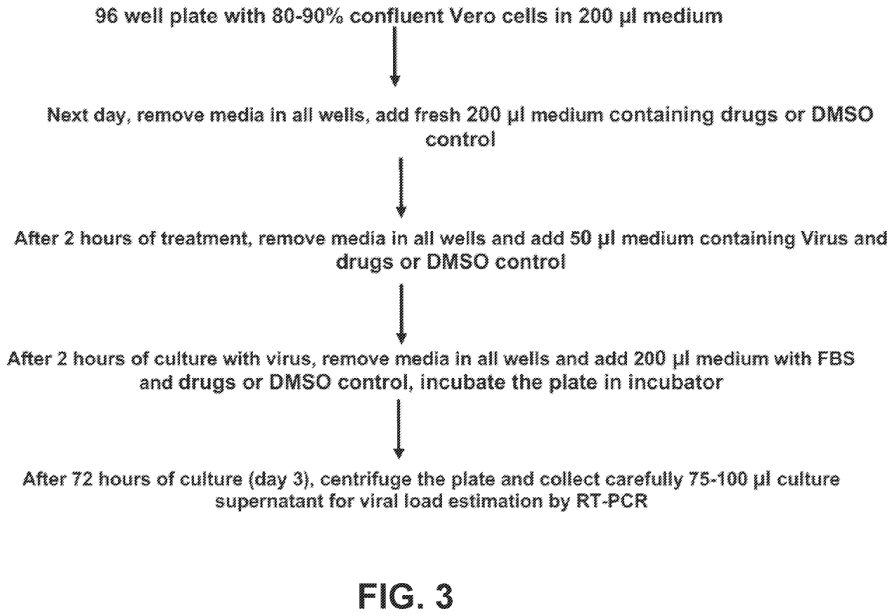

All petri dishes, dilution tube racks, and host-containing apparatus were labeled with the following information: virus, host, test agent. Briefly, a flask of Vero cell grown in cell culture media containing 10% fetal bovine serum (FBS) was used. Cells were seeded in 96-well plate one day before experiment, next day medium is removed, cells are treated with test compound for 2 hours in 200 μl medium. After 2 h, medium was removed and then cells were infected with SARS-COV-2 at a multiplicity of infection (MOI) of 0.01 in the presence of same concentrations of test compound or PBS control. The dose-response curves are determined by quantification of viral RNA copy numbers in the supernatant of infected cell at 72 h post infection (p.i.). A summary of the experimental workflow is provided in FIG. 3.

The test compound is as given below.

### Prolectin-I

### IV Solution of a Single Ingredient:

1. P-01 at a concentration of 1 mg/ml. A vial containing 20 ml and is administered as 4 vials or 80 mg for a 70 kg adult-40 mg/m2 dosing.

### Acceptance Criteria

The experiment was done in duplicates and the values were averaged to calculate % viral reduction. The regression equation for viral particles Vs Ct value of the N-gene specific to SARS-COV-2 virus (y=3.5422x+4.786, R²=0.99) (X=Number of viral particles, y=ctValue) Number of particles are calculated using, X=(40.786−Ct_{Rd/Rp-genes@different times points})/3.5422. Any log reduction in the % viral particles in the experiments is considered to an efficacy of the test compounds to block viral infectivity to the vero cells. Viral reduction was calculated using the following formula:

$$\% of viral reduction = \frac{\begin{matrix}( number of viral particles in infection control - \\ number of viral particles exposed to test drugs )\end{matrix}}{Number of viral particles in infection control}$$

Table 1 below gives a summary of the results of the experiments. Protocol 1 and 2 provided nearly 2 log reduction in viral copy numbers when compared to control. Treating vero cells with drug before infecting with virus (Protocol 1) and Culturing the vero cells with virus and later treating with test drugs demonstrated a near 2 log reduction (99% is a 2log reduction) in viral copy numbers.

TABLE 1
Summary of Results

| | | | Test compounds |
|---|---|---|---|
| | | | compared to Control |
| | | Range of Dose | (% of viral load |
| Test Compound | Experiment | concentration | reduction) |
| Prolectin-I | Protocol 1 - Vero cells | 6.25 μg/ml, | 98%, |
| | in plate treated with | 12.50 μg/ml, | 95%, |
| | Sars-Cov-2 virus and | 25 μg/ml, | 95%, |
| | test drugs/control | 50 μg/ml, | 95%, |
| | (DMSO) | 100 μg/ml, | 95%, |
| | | 200 μg/ml | 89%, |
| Prolectin-I | Protocol 1 - Vero cells | 6.25 μg/ml, | 97%, |
| | in plate treated with | 12.50 μg/ml, | 96%, |
| | Sars-Cov-2 virus and | 25 μg/ml, | 95%, |
| | test drugs/control | 50 μg/ml, | 91%, |
| | (DMSO) | 100 μg/ml, | 94%, |
| | | 200 μg/ml | 89%, |

### Protocol 1.

### Assay Details:

TABLE 2
Assay Details for Protocol 1 Experiment

| Assay Performed | Materials Used | Manufacturer |
|---|---|---|
| Viral RNA Extraction | MaxMAX ™ Viral/Pathogen II | Applied Biosystems (by Thermo |
| | Nucleic Acid Isolation Kit | Fisher Scientific |
| qRT-PCR | Q-line ® Molecular nCOV19 RT- | POCT Services Private Limited |
| | PCR Detection Kit (Multiplex) | |

Tested Concentrations (μg/mL) 6.25, 12.5, 25, 50, 100, 200.

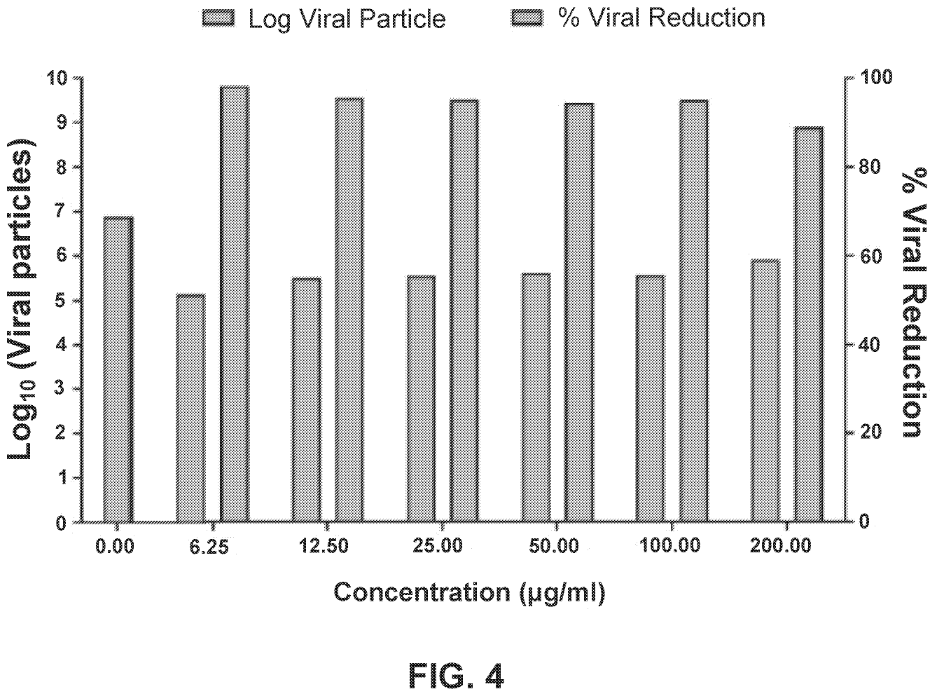

Results: The Prolectin-I showed 98, 95, 95, 95, 95, and 89% viral reduction from 6.25 μg/mL until 200 μg/mL. The viral particles reduced from 106.8 to 105.1. The experiment was done in duplicates and the values were averaged to calculated % viral reduction. The regression equation for viral particles Vs Ct value of the N-gene specific to SARS-COV-2 virus (y=−3.5422x+40.786, R²=0.99)(X=Number of viral particles, y=Ct value). Number of viral particles are calculated using X=(40−0.786−Ct_{Rdrp-gene@different time points})/3.5422 (FIG. 4).

$$\% Viral reduction = \frac{\begin{matrix}number of viral particles in infection control - \\ number of viral particle exposed to drug ( test )\end{matrix}}{number of viral particles in infection control} \times 100$$

### Protocol 2.

### Assay Details:

TABLE 3
Assay Details for Protocol 2 Experiment

| Assay Performed | Materials Used | Manufacturer |
|---|---|---|
| Viral RNA Extraction | MaxMAX ™ Viral/Pathogen II | Applied Biosystems (by Thermo |
| | Nucleic Acid Isolation Kit | Fisher Scientific |
| qRT-PCR | Q-line ® Molecular nCOV19 RT- | POCT Services Private Limited |
| | PCR Detection Kit (Multiplex) | |

Tested Concentrations (μg/mL 6.25, 12.5, 25, 50, 100, 200.

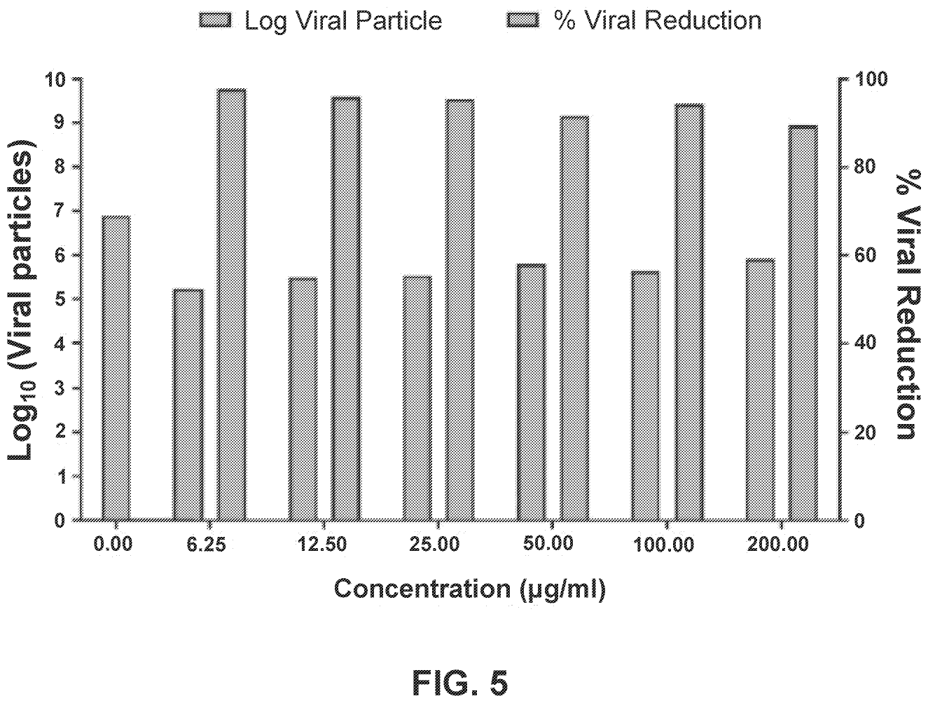

The Prolectin-I showed 97, 96, 95, 91, 94, and 89% viral reduction from 6.25 μg/mL until 200 μg/mL. The viral particles reduced from 10^{6.8} to 10^{5.2.} The experiment was done in duplicates and the values were averaged to calculated % viral reduction. The regression equation for viral particles Vs Ct value of the N-gene specific to SARS-COV-2 virus (y=−3.5422x 40.786, R²=0.99) (X=Number of viral particles, y=Ct value). Number of viral particles are calculated using X=(40.786−Ct_{Rdrp-gene@different time points)}/3.5422) (FIG. 5).

$$\% Viral reduction = \frac{\begin{matrix}number of viral particles in infection control - \\ number of viral particles exposed to drug ( test )\end{matrix}}{number of viral particles in infection control} \times 100$$

### Example 2. NMR Analysis of Prolectin-I

### Glycosyl Linkage Analysis

Prolectin-I solution from two sample vials (total 40 mL) was dialyzed in a dialysis tubing (COMW, 3.5 kDa) against nanopure water (total 4×3 L) for 2 days. The dialyzed sample was lyophilized. Glycosyl linkage analysis was performed by combined gas chromatography/mass spectrometry (GC/MS) of the partially methylated alditol acetates (PMAAs) derivatives produced from the sample. The procedure was a slight modification of the one described by Willis et al. (2013) PNAS, 110 (19) 7868-7873.

Briefly, methylation of the sample using dimsyl potassium base was performed. This was followed by acetylation using N-methylimidazole and acetic anhydride. The sample was extracted with dichloromethane, and the carboxylic acid methyl esters were reduced using lithium aluminum deuteride in THF (80° C., 8 h). After desalting using an On-guard H⁺ column (ThermoFisher), the sample was remethylated using two rounds of treatment with sodium hydroxide (15 min each) and methyl iodide (45 min each). The sample was then hydrolyzed using 2 M TFA (2 h in sealed tube at 120° C.), reduced with NaBD₄, and acetylated using acetic anhydride/TFA. The resulting PMAAs were analyzed on an Agilent 7890A GC interfaced to a 5975C MSD (mass selective detector, electron impact ionization mode); separation was performed on a 30 m Supelco SP-2331 bonded phase fused silica capillary column.

TABLE 4
GC temperature program for linkage analysis

| | Rate (° C./min) | Value (° C.) | Hold Time (min) |
|---|---|---|---|
| Initial | | 60 | 1 |
| Ramp1 | 27.5 | 170 | 0 |
| Ramp2 | 4 | 235 | 2 |
| Ramp3 | 3 | 240 | 12 |

### NMR Analysis

Sample Prolectin-1 7.24 mg was weighed and dissolved in 600 μl of D₂O. The supernatant was transferred into an NMR tube. Ten microliters of 1 mM DSS was added into the NMR tube as internal standard. The sample was analyzed at 25° C. with a Bruker 900 MHz NMR instrument equipped with a cryoprobe. A standard zgf2pr pulse sequence was employed with a pre-saturation sequence for water suppression. Pulse sequences, hsqcetgpsisp2 and clmlevphpr, were applied for collecting HSQC and TOCSY spectrum, respectively.

### Results

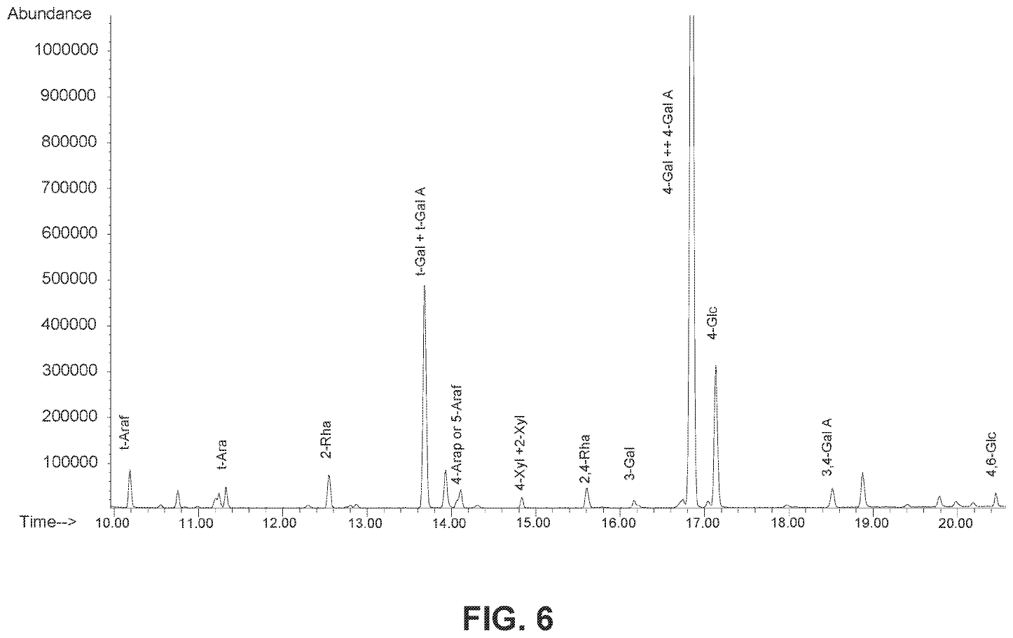

The glycosyl linkage analysis chromatogram is shown in FIG. 6, and the results are listed in Table 5. In summary, the most abundant glycosyl residues of Prolectin-I were GalpA residues (45.7%), including 4-GalpA (34.9%), 3,4-GalpA (1.3%), and t-GalpA (9.5%). In addition, 2-Rhap & 2,4-Rhap residues accounted for 3.4% of the glycosyl residues. This suggested that Prolectin-I has a pectin structure, which is mainly composed of homogalacturonan (HG) with short rhamnogalacturonan-I (RG-I) fragment(s). Moreover, the presence of t-Galp (5.7%) and 4-Galp (30.0%) indicated that the RG-I part of Prolectin-I pectin has β-1,4-galactan side chains. Other glycosyl residues such as t-Araf, t-Arap, 4-Arap/5-Araf, and 3,6-Galp could be minor substituted glycosyl residues.

TABLE 5
Relative percentage of each detected linkage in Prolectin-I.

| Glycosyl Linkage Residue | Relative Area Percentage (%) |
|---|---|
| Terminal Arabinofuranosyl Residue (t-Araf) | 1.9 |
| Terminal Arabinopyranosyl Residue (t-Arap) | 1.1 |
| 2-linked Rhamnopyranosyl Residue (2-Rhap) | 2.0 |
| Terminal Galactopyranosyl Residue (t-Galp) | 5.7 |
| Terminal Galactopyranosyl Uronic Acid Residue (t- | 9.5 |
| GalpA) | |
| 4-linked Arabinopyranosyl or 5-linked | 1.0 |
| Arabinofuranosyl Residue (4-Arap or 5-Araf) | |
| 2-linked Xylopyranosyl Residue (2-Xylp) | 0.4 |
| 4-linked Xylopyranosyl Residue (4-Xylp) | 0.2 |
| 2,4-linked Rhamnopyranosyl Residue (2,4-Rhap) | 1.4 |
| 3-linked Galactopyranosyl Residue (3-Galp) | 0.4 |
| 4-linked Galactopyranosyl Residue (4-Galp) | 30.0 |
| 4-linked Galactopyranosyl Uronic Acid Residue (4- | 34.9 |
| GalpA) | |
| 4-linked Glucopyranosyl Residue (4-Glcp) | 9.6 |
| 3,4-linked Galactopyranosyl Uronic Acid Residue | 1.3 |
| (3,4-GalpA) | |
| 3,6-linked Galactopyranosyl Residue (3,6-Galp) | 0.7 |
| Total | 100.1 |

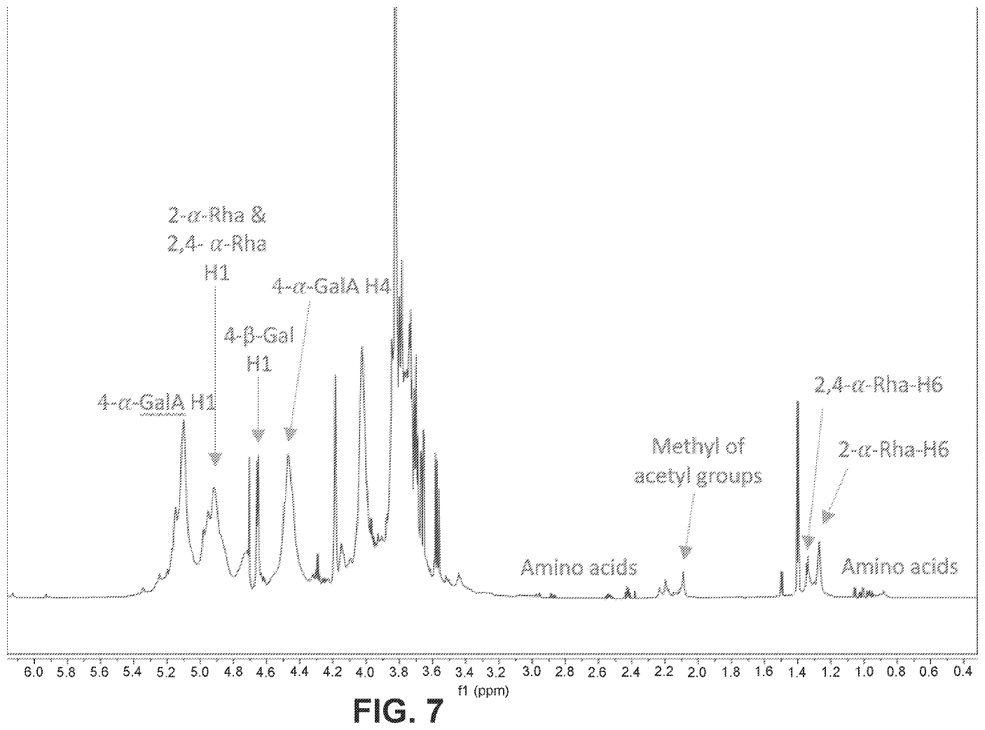

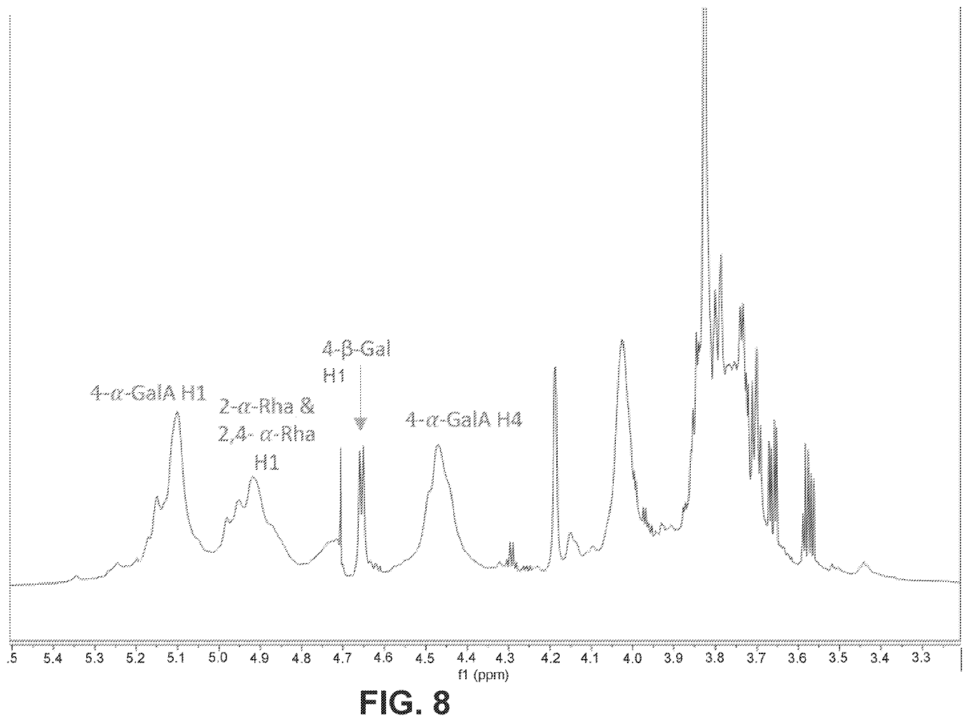

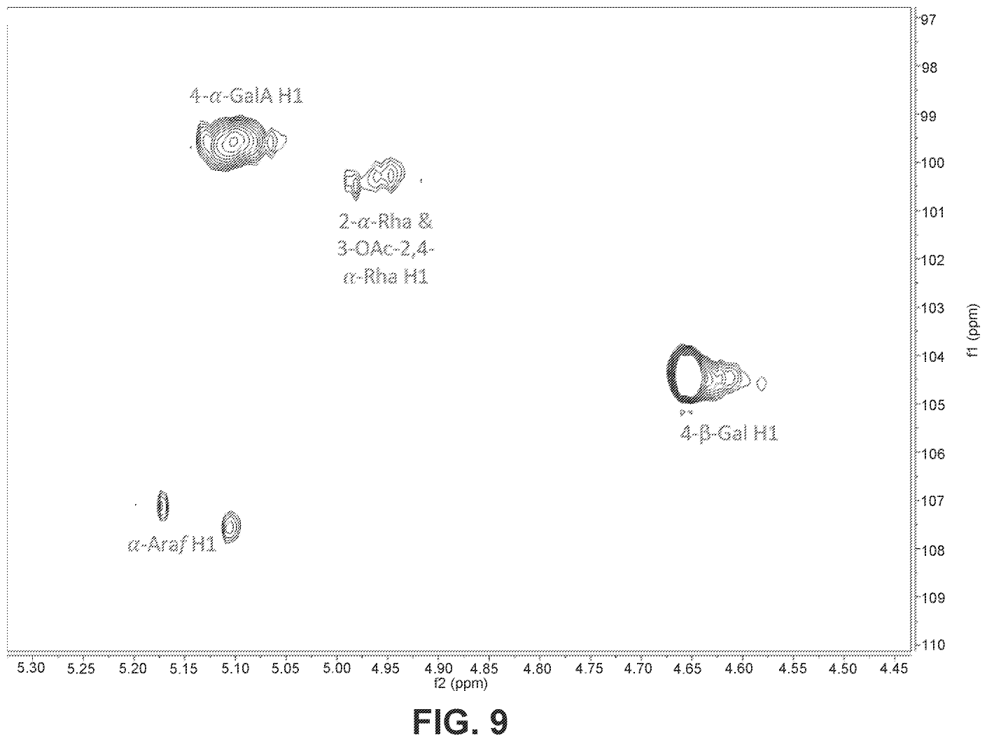

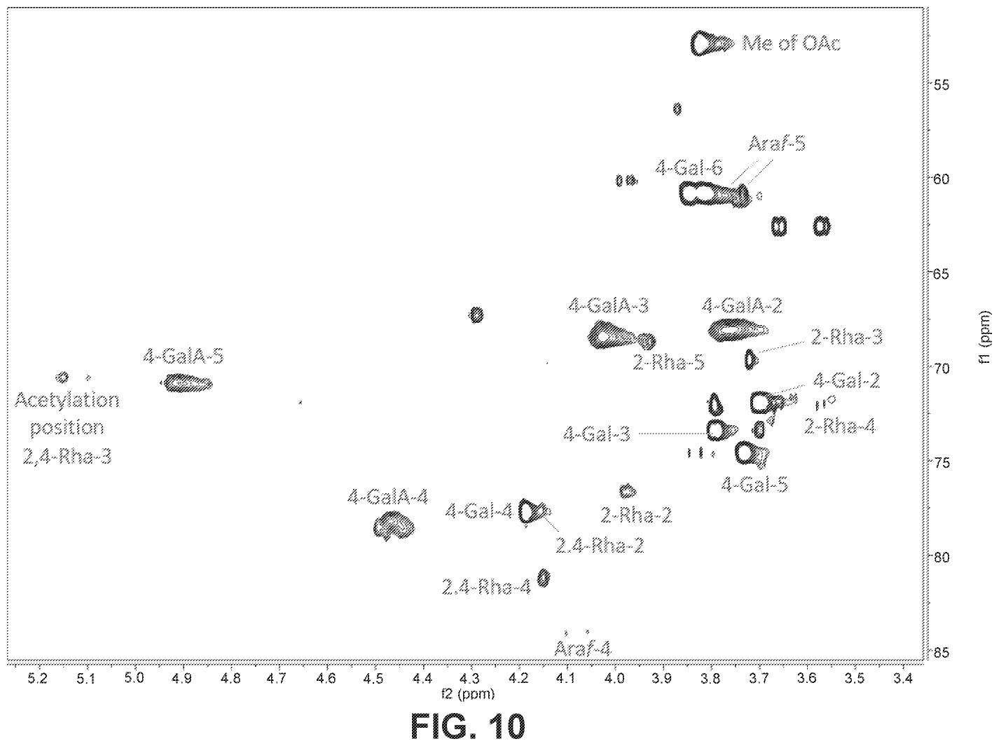

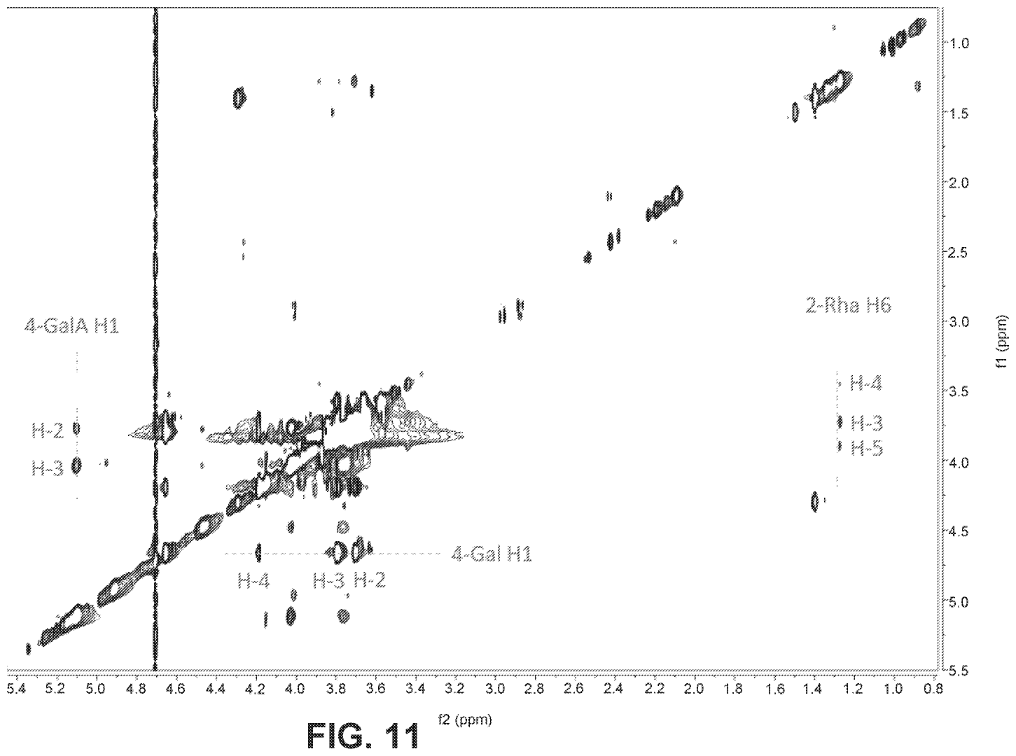

The NMR spectra are shown in FIGS. 5-9. The 1D ¹H-NMR spectrum showed that the GalA and Rha had alpha-configurations, while Gal residues have beta-configurations (FIGS. 7 and 8). The ¹H/¹³C cross-peaks were assigned and labeled in FIG. 9 and FIG. 10. The chemical shifts of the major glycosyl residues are summarized in Table 6. The chemical shift assignments were based on a TOCSY spectrum (FIG. 11) and published data (see references below). In summary, the NMR results supported the linkage analysis data; both confirmed the major component of Prolectin-I is pectin, composed of HG and B-1,4-galactan-containing RG-I.

TABLE 6
Chemical shift assignment of major glycosyl residues of Prolectin-I.

| Glycosyl | C/H-1 | C/H-2 | C/H-3 | C/H-4 | C/H-5 | C/H-6 |
|---|---|---|---|---|---|---|
| α-Araf | 107/5.17 | n.d. | n.d. | 84.1/4.10 | n.d. | — |
| α-Araf | 107/5.11 | n.d. | n.d. | 84.1/4.06 | n.d. | — |
| 4-α-GalA | 99.6/5.10 | 68.0/3.76 | 68.5/4.03 | 78.2/4.47 | 70.9/4.91 | — |
| 2-α-Rha | 100/4.96 | 76.6/3.98 | 69.7/3.72 | 71.8/3.55 | 68.7/3.94 | 16.6/1.26 |
| 3-OAc- | 100/4.96 | 77.7/4.15 | 70.6/5.15 | 81.1/4.15 | — | 19.7/1.34 |
| 2,4-α-Rha | | | (OAc: 52.9/3.82) | | | |
| 4-β-Gal | 104/4.66 | 71.9/3.70 | 73.4/3.79 | 77.7/4.19 | 74.6/3.73 | 60.9/3.85, |
| | | | | | | 3.81 |

Prolectin-I also contained weak signals of amino acids (FIG. 7 and FIG. 10), as well as about 9.6% of 4-Glcp as identified in the glycosyl linkage analysis (Table 5).

### REFERENCES

Bushneva O.A., Ovodova R.G. Shashkov A.S., Chizhov A.O., Ovodov Y.S. (2003) Structure of Silenan, a pectic polysaccharide form campion Silene vulgaris (Moench) Garcke. Biochemistry (Moscow) 68: 1360-1368
Huisman M.M.H., Fransen C.T.M., Kamerling J.P., Vliegenthart J.F.G., Schols H.A., Voragen A.G.J. (2001) The CDTA-soluble pectic substances from soybean meal are composed of rhamnogalacturonan and xylogalacturonan but not homogalacturonan. Biopolymers 58: 279-294
Sengkhamparn N., Bakx E.J., Verhoef R., Shols H.A., Sajaanantakul T., Voragen A.G.J. (2009) Okra pectin contains an unusual substitution of its rhamnosyl residues with acetyl and alpha-linked galactosyl groups. Carbohydrate Research 344: 1842-1851

Other embodiments are in the claims.

1. A method of treating a SARS-COV-2 infection in a subject, the method comprising administering to the subject an effective amount of pectin polysaccharides.

2. The method of claim 1, wherein administration of pectin polysaccharides to the subject reduces the SARS-COV-2 infectivity of the subject.

3. The method of claim 1, wherein administration of pectin polysaccharides decreases the copy number of SARS-COV-2 RNA polynucleotides present in a biological sample from the subject.

4. The method of claim 3, wherein the biological sample from the subject is nasal secretions, blood, saliva, serum, and/or stool.

5. The method of claim 1, wherein administration of the effective amount of pectin polysaccharides increases the copy number of a SARS-COV-2 gene.

6. The method of claim 5, wherein the SARS-COV-2 gene is the SARS-COV-2 envelope protein gene or the SARS-COV-2 nucleocapsid gene or the SARS-COV-2 RNA-dependent RNA polymerase gene.

7. (canceled)

8. (canceled)

9. The method of claim 5, wherein the copy number of the SARS-COV-2 gene is measured in a real time polymerase chain reaction experiment.

10. The method of claim 5, wherein the copy number of the SARS-COV-2 gene is measured using a biological sample obtained from the subject.

11. (canceled)

12. The method of claim 1, wherein administration of the effective amount of pectin polysaccharides results in an increase in the Immunoglobulin G antibody titer in the subject.

13. The method of claim 1, wherein the pectin polysaccharides are administered intravenously.

14. The method of claim 1, wherein the pectin polysaccharides are administered to the subject at least one time a day.

15. The method of claim 14, wherein the pectin polysaccharides are administered to the subject at least two times a day

16. The method of claim 14, wherein the pectin polysaccharides are administered to the subject up to ten times a day.

17. The method of claim 15, wherein the pectin polysaccharides are administered to the subject one time per hour during each hour that the subject is awake.

18. A method of reducing the number of SARS-COV-2 virons in a sample of cells, the method comprising contacting the sample with an effective amount of pectin polysaccharides.

19. The method of claim 18, wherein the number of virons in the sample of cells is reduced by at least 50%.

20. The method of claim 19, wherein the number of virons in the sample of cells is reduced by at least 80%.

21. The method of claim 20, wherein the number of virons in the sample of cells is reduced by at least 95%.

22. The method of claims 18, wherein the sample of cells is exposed to SARS-COV-2 virons prior to being contacted with the effective amount of pectin polysaccharides.

23. The method of claim 18, wherein the sample of cells is contacted with the effective amount of pectin polysaccharides prior to being exposed to SARS-COV-2 virons.
